(19) United States
(12) Patent Application Publication
(10) Pub. No.: US 2019/0038340 A1
(43) Pub. Date: Feb. 7, 2019

(54) RADIOFREQUENCY CANNULA WITH FENESTRATED ACTIVE TIP

(71) Applicant: Jaspal Ricky SINGH, New York, NY (US)

(72) Inventor: Jaspal Ricky SINGH, New York, NY (US)

(21) Appl. No.: 15/824,123

(22) Filed: Nov. 28, 2017

Related U.S. Application Data
(60) Provisional application No. 62/540,236, filed on Aug. 2, 2017.

(51) Int. Cl. A61B 18/14; A61N 1/372

(52) U.S. Cl. CPC A61B 18/148; A61N 1/36071; A61N 1/37205

(57) ABSTRACT

Devices and methods for radio frequency ablation (RFA) are disclosed. A radiofrequency cannula may comprise a cannula body configured to deliver anesthetic and/or electric current therethrough, and an active tip located at a distal tip of the cannula body. The active tip may comprise one or more fenestrations configured to pass anesthetic laterally from at least one side of the active tip.

The present application for patent claims the benefit of U.S. Provisional Application No. 62/540,236, entitled “RADIOFREQUENCY CANNULA WITH FENESTRATED ACTIVE TIP”, filed 2 Aug. 2017, assigned to the assignee hereof, and expressly incorporated herein by reference in its entirety. Aspects of this disclosure relate generally to a radiofrequency cannula with a fenestrated active tip.

### INTRODUCTION

A cannula is a thin, insulated tube inserted into the body of the patient to administer medicine, drain fluid or insert a surgical instrument. There is a pain treatment technique known as “Radiofrequency Neurotomy” or “Radiofrequency Ablation” (sometimes abbreviated as “RFA”). In RFA, the cannula is a radiofrequency cannula having an active tip that radiates heat. First, the radiofrequency cannula may be inserted into a body of a patient, in particular, an area associated with pain (for example, the spine). After the radiofrequency cannula is positioned in the proximity of the area to be treated, anesthetic is injected through the cannula, which numbs tissue within the surgical area. A radiofrequency probe is then inserted through the cannula. When a current is applied to the radiofrequency probe, the active tip heats up tissue within the surgical area. As the tissues heat up, the nerves are denatured and as a result, pain signals transmitted by these nerves are abolished or reduced.

Skill is required to perform RFA. For example, the radiofrequency cannula must be guided to a precise area, and the proper positioning of the radiofrequency cannula must be verified. After the area is treated, the procedure may be performed again on a different area. Accordingly, new tools and methods are required in order to improve the speed, efficacy, reliability and patient comfort of RFA.

### SUMMARY

Devices and methods for RFA are disclosed. The following summary is an overview provided solely to aid in the description of various aspects of the disclosure and is provided solely for illustration of the aspects and not limitation thereof.

In accordance with aspects of the disclosure, a radiofrequency cannula is disclosed. The radiofrequency cannula comprises, for example, a cannula body configured to deliver anesthetic and/or electric current therethrough, and an active tip located distally with respect to the cannula body. The active tip may comprise one or more fenestrations configured to pass anesthetic laterally from at least one side of the active tip.

In accordance with other aspects of the disclosure, a method is disclosed. The method comprises, for example, maneuvering a cannula body having an active tip located distally with respect to the cannula body to a treatment site having a target area, wherein the active tip comprises one or more fenestrations configured to pass anesthetic laterally from at least one side of the active tip, delivering anesthetic laterally to the target area via the cannula body and through the one or more fenestrations, inserting a radiofrequency probe through the cannula body such that it contacts the active tip, and activating the radiofrequency probe to ablate the target area.

In accordance with other aspects of the disclosure, another radiofrequency cannula is disclosed. The radiofrequency cannula comprises, for example, means for passing anesthetic and/or electric current therethrough, and means for delivering anesthetic and/or electric current located distally with respect to the means for passing, wherein the means for delivering comprises one or more fenestrations configured to pass anesthetic laterally from at least one side of the active tip.

### BRIEF DESCRIPTION OF THE DRAWINGS

The accompanying drawings are presented to aid in the description of various aspects of the disclosure and are provided solely for illustration of the aspects and not limitation thereof.

FIG. 1 generally illustrates a surgical area in which RFA may be performed.

FIG. 2A generally illustrates a conventional curved cannula with a sharp tip.

FIG. 2B generally illustrates a conventional curved cannula with a blunt tip.

FIG. 2C generally illustrates a conventional straight cannula with a sharp tip.

FIG. 3 generally illustrates an anesthesia treatment in accordance with a conventional RFA technique.

FIG. 4 generally illustrates an ablation treatment in accordance with the conventional RFA technique depicted in FIG. 3.

FIG. 5 generally illustrates a cannula with a fenestrated active tip in accordance with aspects of the disclosure.

FIG. 6 generally illustrates another cannula with a fenestrated active tip in accordance with aspects of the disclosure.

FIG. 7 generally illustrates yet another cannula with a fenestrated active tip in accordance with aspects of the disclosure.

FIG. 8 generally illustrates yet another cannula with a fenestrated active tip in accordance with aspects of the disclosure.

FIG. 9 generally illustrates an anesthesia treatment and an ablation treatment in accordance with aspects of the disclosure.

### DETAILED DESCRIPTION

The cannulas that are conventionally used in RFA procedures fail to accurately distribute anesthesia throughout an ablation target area. The inaccuracy is the result of the conventional cannula design. In particular, anesthesia is conventionally provided through a distal opening in the cannula. As a result, the anesthetized area is distal to and somewhat beyond the active tip of the cannula. By contrast, the ablation is performed around a circumference of the active tip, along the entirety of the length of the active tip. There may be significant overlap between the anesthetized area and the ablated area, but there is also significant anesthetization of non-targeted tissue (which can cause numbness in the recovering patient), and significant ablation of non-anesthetized tissue (which can cause pain during the RFA procedure).

The present disclosure relates to a redesigned cannula that maximizes the coincidence between the anesthetized area and the ablated area. To ensure an even distribution of anesthesia around the active tip of the cannula, fenestrations are provided on at least one side of the active tip. Anesthesia may be delivered through the fenestrations. As a result, the risk of unnecessary anesthetization of non-ablated area is reduced, as is the risk of ablation of non-anesthetized tissue. Exemplary implementation details are disclosed in greater detail below.

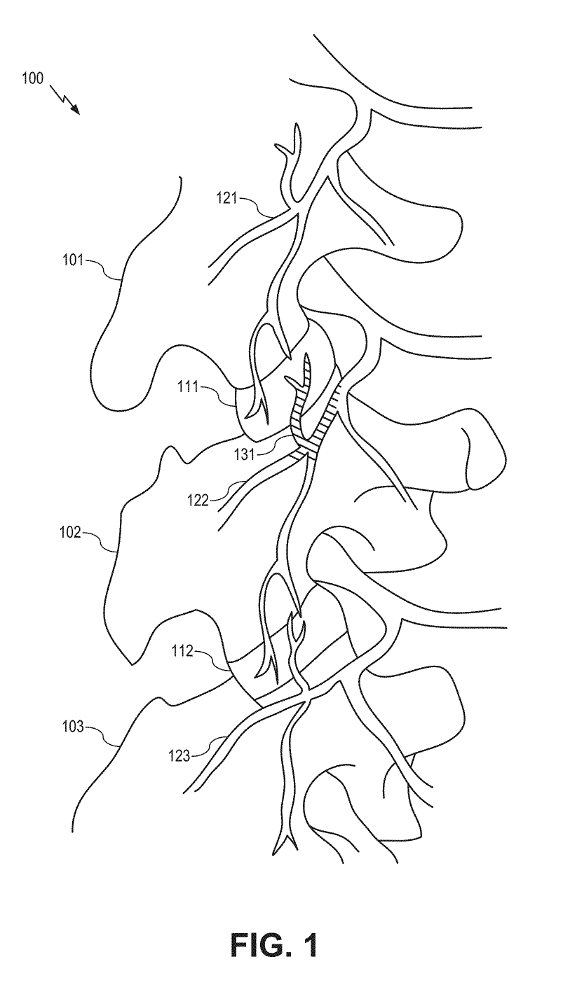

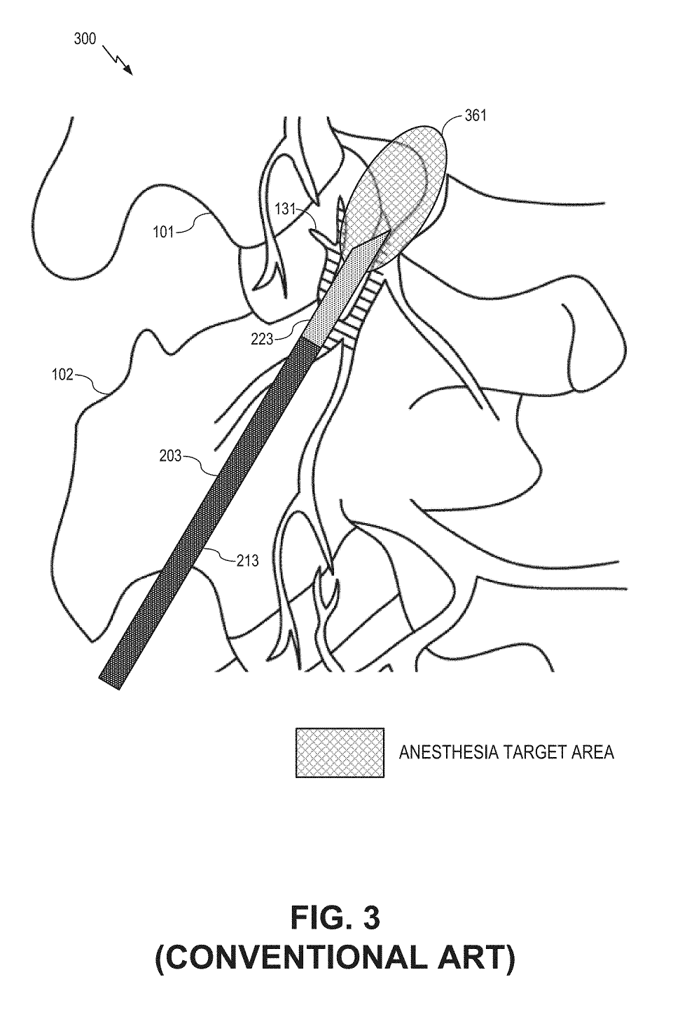

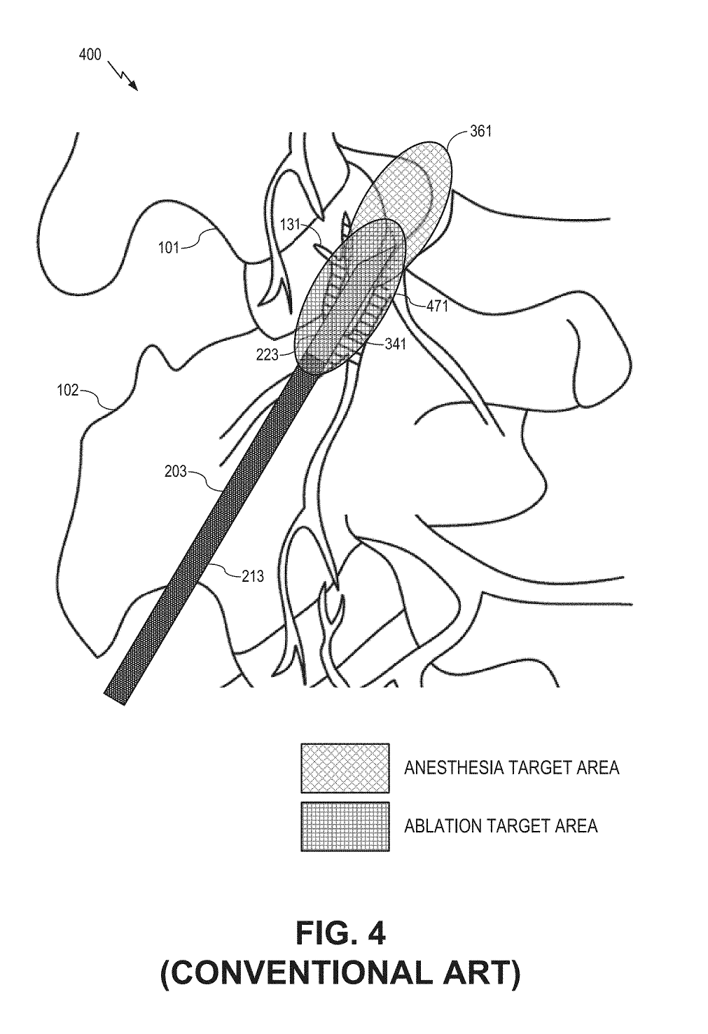

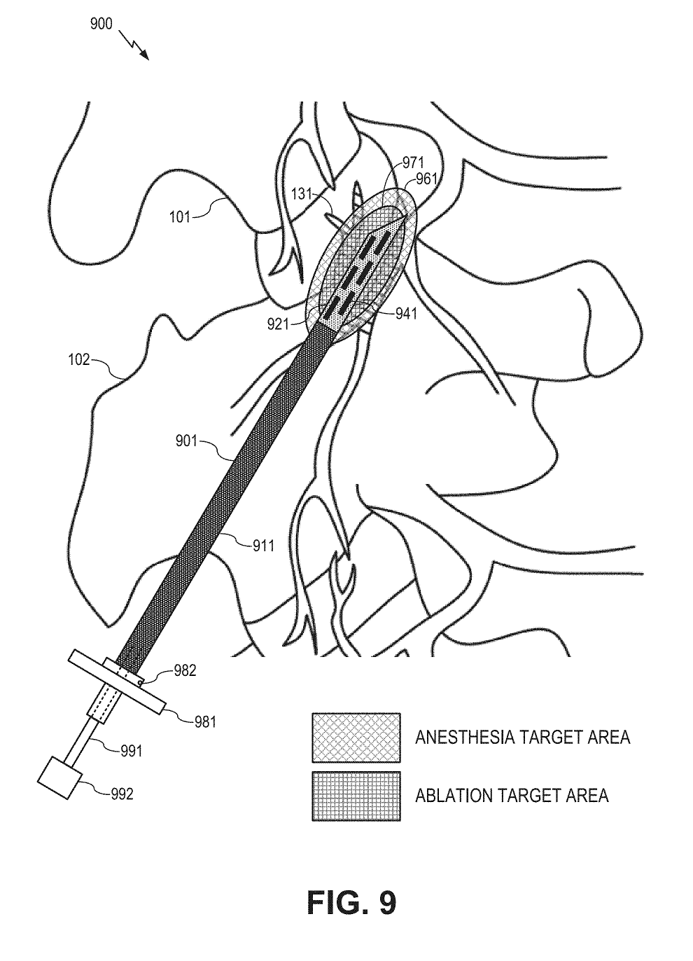

FIG. 1 generally illustrates a surgical area 100 in which RFA may be performed. FIGS. 3-4 depicts a conventional cannula performing the RFA technique within the surgical area 100, and FIG. 9 depicts a cannula in accordance with aspects of the disclosure performing the RFA technique in the surgical area 100. FIGS. 3-4 and 9 will be discussed in greater detail below.

In the example of FIG. 1, the surgical area 100 includes a portion of a human vertebral column, also known as a spine or backbone. The depicted portion of the vertebral column includes a vertebra 101, a vertebra 102, and a vertebra 103 in a stacked configuration. In particular, the vertebrae 101-103 are joined to one another in a column. The vertebral column has a spinal canal running from top to bottom, which guides and protects a spinal cord. The spinal cord carries nerve impulses throughout the human body.

The vertebral column further includes a facet joint 111 between the bottom of the vertebra 101 and the top of the vertebra 102 and a facet joint 112 between bottom of the vertebra 102 and top of the vertebra 103. The vertebral column further includes a medial nerve branch 121 on the vertebra 101, a medial nerve branch 122 on the vertebra 102, and a medial nerve branch 123 on the vertebra 103. The medial nerve branches 121-123 emerge from the spinal cord and sense pain caused by trauma or stress of the vertebral column.

In FIG. 1, the vertebral column includes a target nerve 131. The target nerve 131 may be an inflamed portion of the medial nerve branch 122 that is causing localized pain for the patient in the area of the vertebrae 101-102. In many cases, the pain may be caused by trauma or stress to some portion of the vertebral column, for example, the facet joint 111. Back pain can be debilitating to the patient. Accordingly, the medical profession has developed techniques for reducing the sensitivity of the medial nerve branches 121-123.

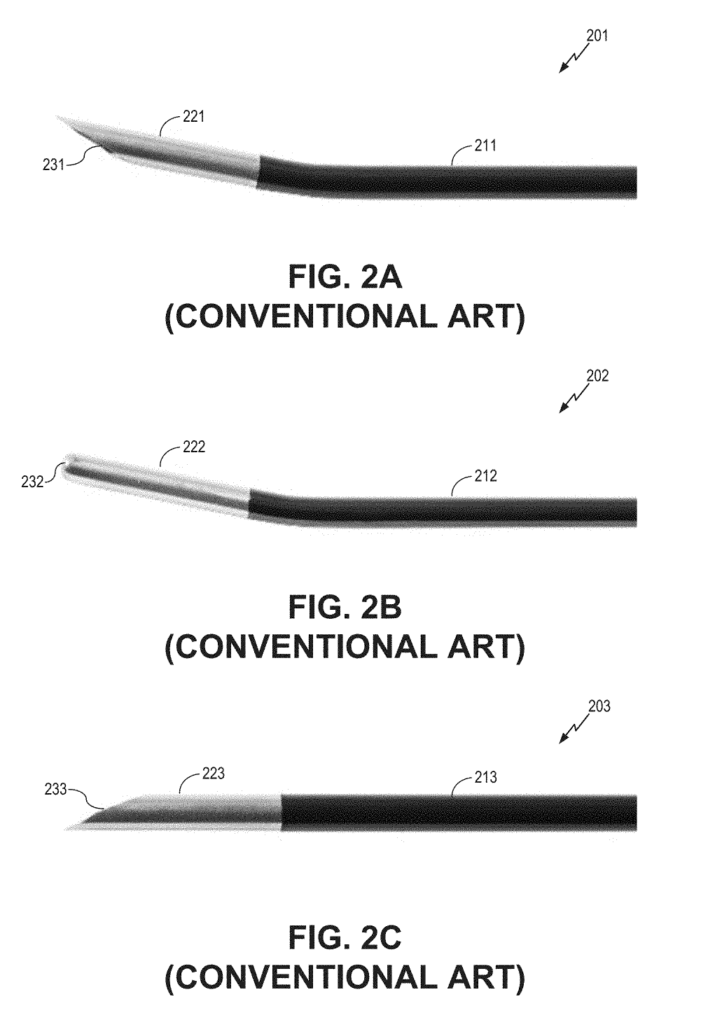

FIG. 2A generally illustrates a conventional curved cannula 201 with a sharp tip. The sharp curved cannula 201 comprises an insulated cannula body 211, an exposed active tip 221, and a distal end 231. The cannula body 211 may be a hollow insulating tube. The active tip 221 is configured to radiate heat generated by a wire probe in response to application of an electric current. The distal end 231 may have a distal opening for permitting fluid flow into or out of the sharp curved cannula 201.

FIG. 2B generally illustrates a conventional curved cannula 202 with a blunt tip. The blunt curved cannula 202 comprises a cannula body 212, an active tip 222, and a distal end 232. The cannula body 212 and the active tip 222 may be analogous to the cannula body 211 and the active tip 221 depicted in FIG. 2A. However, unlike the distal end 231 depicted in FIG. 2A, the distal end 232 depicted in FIG. 2B is blunt.

FIG. 2C generally illustrates a conventional straight cannula 203 with a sharp tip. The sharp straight cannula 203 comprises a cannula body 213, an active tip 223, and a distal end 233. Unlike the sharp curved cannula 201 depicted in FIG. 2A, the sharp straight cannula 203 depicted in FIG. 2C has a straight cannula body 213. However, the cannula body 213, the active tip 223, and the distal end 233 are otherwise analogous to the cannula body 211, the active tip 221, and the distal end 231 depicted in FIG. 2A.

Each of the conventional cannulas 201-203 depicted in FIGS. 2A-2C may be used to perform surgical pain treatment. It will be understood that other arrangements of the cannulas 201-203 are possible, for example, sharper or straighter curves, sharper or blunter distal ends, etc. However, commercially available cannulas are similar in that they typically have a single opening at a distal extent of the cannula.

FIG. 3 generally illustrates a surgical area 300 in which an anesthesia treatment is performed in accordance with a conventional RFA technique.

In FIG. 3, a portion of the vertebral column is shown once again, including the vertebra 101, the vertebra 102, the medial nerve branch 122, and the target nerve 131 described above in relation to FIG. 1. FIG. 3 also includes the sharp straight cannula 203 depicted in FIG. 2C, which comprises the cannula body 213, the active tip 223, and the distal end 233.

In a conventional RFA technique, the target nerve 131 may be anesthetized prior to ablation. To perform the anesthesia, a liquid anesthetic is directed through the cannula body 213 in the direction of the distal end 233. Once it reaches the distal end 233, it is injected into the surgical area 300, in particular, an anesthesia target area 361.

It will be understood that the anesthesia target area 361 will be located distally with respect to the cannula 203. If the surgeon intends to ablate as much of the target nerve 131 as possible, then the active tip 223 must be positioned in close proximity to the target nerve 131. However, for conventional cannulas like the cannula 203, proper positioning of the active tip 223 for ablation purposes may prevent accurate administration of the anesthesia. For example, when the surgeon maneuvers the cannula 203 to a position at which it can accurately ablate the target nerve 131, it is unlikely to be in a position where it can fully anesthetize the target nerve 131. Because the anesthesia is distributed distally from the cannula 203, the anesthesia target area 361 may be positioned beyond the target nerve 131, and may in some cases affect collateral tissue. For example, the anesthesia can affect the ventral root, leading to leg numbness in the patient.

FIG. 4 generally illustrates a surgical area 400 in which an ablation treatment is performed in accordance with the conventional RFA technique described above. The ablation treatment may be performed after the anesthetic treatment depicted in FIG. 3.

In FIG. 4, the same portion of the vertebral column that is depicted in FIG. 3 is shown once again. The anesthesia target area 361 depicted in FIG. 3 is also shown. To perform the ablation, the surgeon inserts a radiofrequency probe through the cannula body 213 until it reaches the active tip 223. When an electrical current is applied to the active tip 223 through the radiofrequency probe, the active tip 223 radiates heat, raising the temperature of the tissue in its proximity. As the tissue heats up, a circumferential lesion is created around the active tip 223. The lesion may cause protein present in the target nerve 131 to be denatured. Accordingly, nociceptive impulses transmitted through the target nerve 131 are blocked. The circumferential lesion is depicted in FIG. 4 as an ablation target area 471.

As will be understood from FIG. 4, the surgeon has maneuvered the cannula 203 such that the target nerve 131 is just beyond the distal end 233 of the sharp straight cannula 203. As a result, the anesthesia target area 361 is accurately distributed over a large portion of the target nerve 131. However, the ablation target area 471 surrounds the active tip 223. As a result, the ablation target area 471 encompasses only a small portion of the target nerve 131. Also problematic is the fact that the ablation target area 471 covers a large amount of tissue other than the target nerve 131. The result is that the ablation treatment is inaccurate. Because the anesthesia target area 361 and the ablation target area 471 have a small area of coincidence, the surgeon is forced to choose whether to accurately perform the anesthetic treatment or to accurately perform the ablation treatment.

As will be discussed in greater detail below, the cannula of the present disclosure enables the surgeon to accurately perform both the anesthetization and the ablation without moving the cannula.

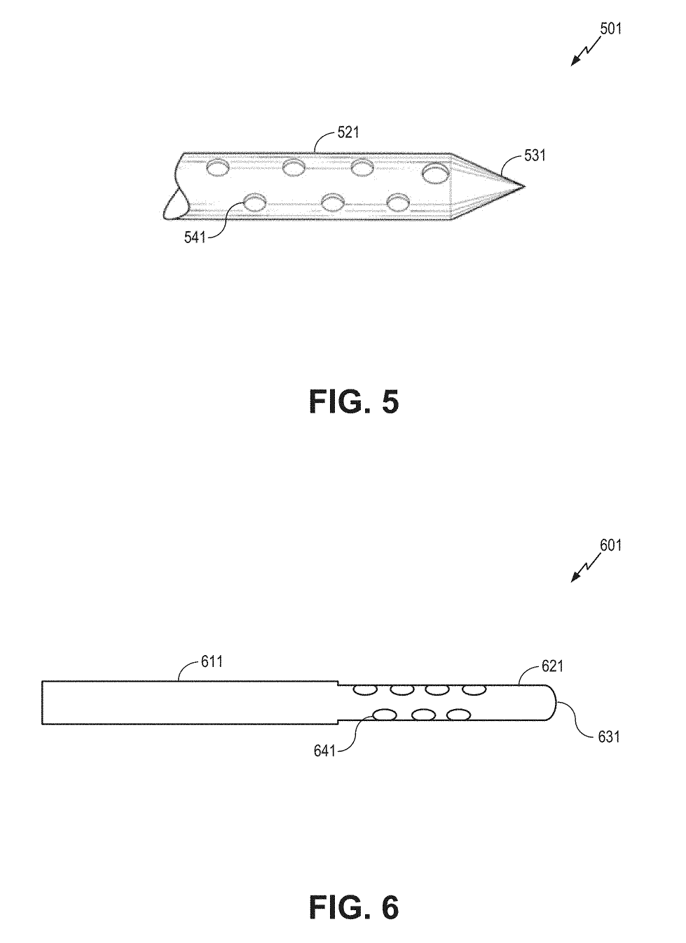

FIG. 5 generally illustrates a cannula 501 with a fenestrated active tip in accordance with aspects of the disclosure. The cannula 501 comprises an active tip 521 and a distal end 531. The active tip 521 is analogous in some respects to the active tips 221-223 depicted in FIGS. 2A-2C. In particular, the active tip 521 is disposed at the end of a cannula body (not shown) and configured to ablate surrounding tissue in response to delivery of an electric current. The distal end 531 is cone-shaped, although other shapes are possible.

Unlike the active tips 221-223 depicted in FIGS. 2A-2C, the active tip 521 includes one or more fenestrations 541. As depicted in FIG. 5, the one or more fenestrations 541 may comprise an ellipse-shaped lateral opening in the active tip 521. The one or more fenestrations 541 may be arranged such that they substantially surround a circumference of the active tip 521. Unlike, for example, the cannula 203 depicted in FIG. 2C, the cannula 501 does not include a distal opening. Instead, the anesthetic is injected through the one or more fenestrations 541. In accordance with aspects of the disclosure, the cannula 501 of the present disclosure may be used to provide anesthesia to an anesthesia target area that surrounds the active tip 521. As a result, there is substantial overlap between the anesthesia target area and the ablation target area.

FIG. 6 generally illustrates another cannula 601 with a fenestrated active tip in accordance with aspects of the disclosure. The cannula 601 comprises a cannula body 611, an active tip 621, a distal end 631, and one or more fenestrations 641. The active tip 621, the distal end 631, and the one or more fenestrations 641 may be analogous in some respects to the active tip 521, the distal end 531, and the one or more fenestrations 541 depicted in FIG. 5, respectively. However, unlike the cannula 501 depicted in FIG. 5, the distal end 631 of the cannula 601 is blunt. In accordance with aspects of the disclosure, the cannula 601 of the present disclosure may be used to provide anesthesia to an anesthesia target area that surrounds the active tip 621. As a result, there is substantial overlap between the anesthesia target area and the ablation target area.

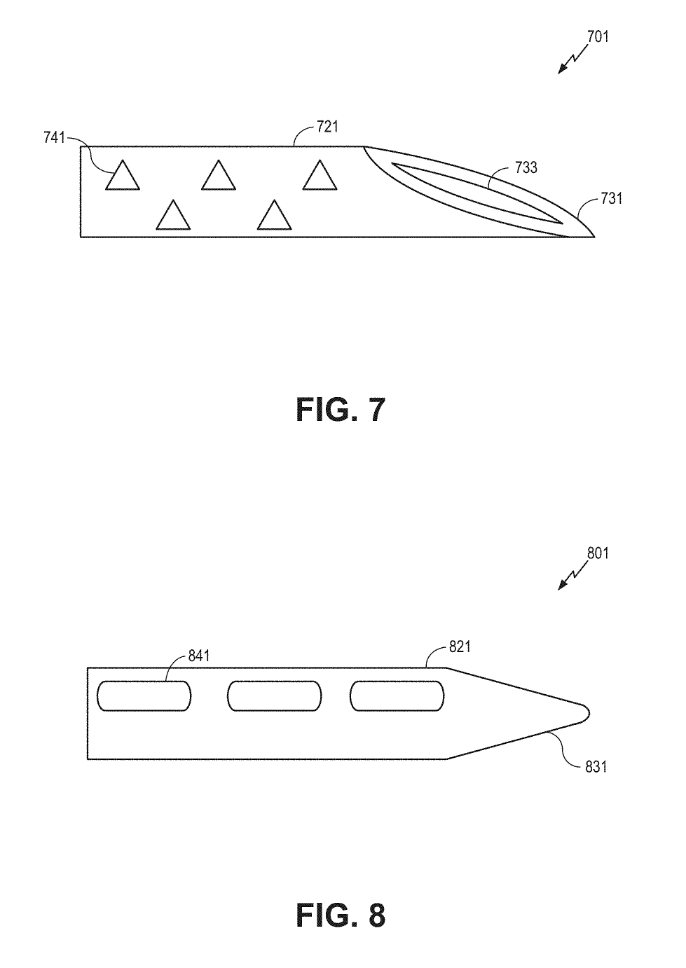

FIG. 7 generally illustrates yet another cannula 701 with a fenestrated active tip in accordance with aspects of the disclosure. The cannula 701 comprises an active tip 721 and a distal end 731, wherein the distal end 731 may be referred to as chisel-shaped. The active tip 721 and the distal end 731 may be analogous in some respects to the active tip 221 and the distal end 231 depicted in FIG. 2C. In particular, the distal end 731 may have a distal opening 733. However, unlike the cannula 203, the cannula 701 further includes one or more fenestrations 741. As depicted in FIG. 7, the one or more fenestrations 741 may comprise triangle-shaped lateral openings in the active tip 721. In accordance with aspects of the disclosure, the cannula 701 of the present disclosure may be used to provide anesthesia to an anesthesia target area that surrounds the active tip 721. As a result, there is substantial overlap between the anesthesia target area and the ablation target area.

FIG. 8 generally illustrates yet another cannula 801 with a fenestrated active tip in accordance with aspects of the disclosure. The cannula 801 comprises an active tip 821 and a distal end 831. The active tip 821 and the distal end 831 may be analogous in some respects to the active tip 521 and the distal end 531 depicted in FIG. 5. The cannula 801 comprises one or more fenestrations 841, which may be linearly arranged on one side of the active tip 821. Unlike the one or more fenestrations 541 depicted in FIG. 5, the one or more fenestrations 841 are slot-shaped lateral openings. In accordance with aspects of the disclosure, the cannula 801 of the present disclosure may be used to provide anesthesia to an anesthesia target area that surrounds the active tip 821. As a result, there is substantial overlap between the anesthesia target area and the ablation target area.

Although FIGS. 5-8 depict several specific arrangements of a cannula in accordance with aspects of the disclosure, it will be understood that other arrangements are possible. For example, the active tip may be of any length, shape, or thickness (for example, in terms of gauge). The active tip may be with or without a distal opening. There may be any number of fenestrations in the active tip, placed at any interval, regular or irregular, along and/or around the active tip. The fenestrations may have the same shape as one another or different shapes.

FIG. 9 generally illustrates a surgical area 900 in which an anesthesia treatment and an ablation treatment are performed with a cannula 901 in accordance with aspects of the disclosure.

Returning briefly to FIG. 4, it will be understood that a conventional cannula such as the cannula 203 does not enable a surgeon to accurately anesthetize a target nerve 131 and then accurately ablate the target nerve 131. This is because the anesthesia target area 361 is located distally with respect to the active tip 223, whereas the ablation target area 471 is located laterally around the active tip 223. As a result, only a portion of the anesthesia target area 361 gets ablated, which suggests that other tissue in the area has been collaterally affected. Sometimes, inaccurately placed anesthetic affects the ventral roots, which can cause numbness in the legs of recovering patients. Moreover, only a portion of the ablation target area 471 has received anesthesia, which can result in pain during the ablation procedure. Accordingly, the effectiveness of the RFA may be reduced.

In FIG. 9, the surgeon uses the cannula 901 in accordance with aspects of the disclosure. The cannula 901 may be similar in some respects to one or more of the cannulas 501-801 depicted in FIGS. 5-8. In particular, the cannula 901 includes a cannula body 911, an active tip 921, a distal end 931, and one or more fenestrations 941. The shape of the active tip 921 may be similar to, for example, the shape of the active tip 721 depicted in FIG. 7. Moreover, the active tip 921 may include multiple rows of linear slits, similar to the one or more fenestrations 841 depicted in FIG. 8, running along a side of the cannula 901.

To begin the procedure, the surgeon guides the cannula 901 through the surgical area 900 until it is proximate (for example, in contact with) the target nerve 131. To maximize the efficiency of the procedure, a length of the active tip 921 is placed parallel to and into contact with the target nerve 131. Successful guidance requires placement of the active tip 921 within the fascial compartment that envelops the target nerve 131.

The cannula 901 may comprise a cannula hub 981 at a proximal end of the cannula body 911. The surgeon may position the cannula 901 by manipulating the cannula hub 981. The cannula hub 981 may include an indicator 982. The indicator 982 may be a shape indicator, a fenestration indicator, or a combination thereof. For example, if the cannula 901 is asymmetrical (for example, having a curved or bent cannula body 911), then the indicator 982 may a point on the circumference of the cannula hub 981 that corresponds to a direction of the asymmetry. For example, with the tip of the cannula 901 curving “upward” (with respect to, for example, gravity), the indicator 982 may be placed such that it is at a “top” or “12 o'clock” position of the cannula hub 981. As the cannula 901 rotates about its longitudinal axis, the direction of the curve will rotate, as will the indicator 982. For example, if the cannula 901 rotates ninety degrees in a clockwise direction, then the tip of the cannula 901 will be curving “rightward” and the indicator 982 will appear at a “3 o'clock” position.

Additionally or alternatively, the indicator 982 may be a fenestration indicator that corresponds to a direction of fluid flow through the one or more fenestrations 941. For example, if the cannula 901 includes a single column of fenestrations (like, for example, the one or more fenestrations 841 depicted in FIG. 8), then the indicator 982 may be placed on the circumference of the cannula hub 981 such that it corresponds to the point on the circumference of the active tip 921 through which fluid flows. For example, if the one or more fenestrations 941 are on a “bottom” of the active tip 921, then the indicator 982 may be placed at the “6 o'clock” position on the cannula hub 981. It will be further understood that if there are multiple rows of the one or more fenestrations 941, then multiple corresponding fenestration indicators may be provided.

It will be understood that if the cannula 901 has a shape indicator and a fenestration indicator, then a plurality of the indicator 982 may be placed on the cannula hub 981. Moreover, if the direction of curvature and the direction of the fluid flow coincide, then a single indicator 982 may constitute a shape indicator and a fenestration indicator.

For the purpose of guiding the cannula 901 to the target nerve 131, a stylet 991 is inserted through the proximal cannula hub 981 and into the cannula body 911. The stylet 991 is a rigid structure that enables the surgeon to advance and maneuver the cannula 901 to the surgical area 900 and proximate to the target nerve 131. The stylet 991 may be made of, for example, metal, plastic, or any other suitable material. The stylet 991 may also be configured to “plug” the one or more fenestrations 941, thereby preventing tissue from getting caught in the one or more fenestrations 941 as the cannula 901 is advanced. Similarly, the stylet 991 may be inserted into the cannula 901 before it is withdrawn from the patient at the conclusion of the procedure. Failure to insert the stylet 991 before withdrawal of the cannula 901 may result in fistulas and/or infections. Withdrawal of the stylet 991 from the cannula hub 981 enables a back flow of fluid into the cannula hub 981 from which proper localization of the cannula 901 may be verified prior to administering the anesthetic.

Once the active tip 921 is in position, the stylet 991 is removed. The stylet 991 may include a stylet cap 992 on a proximal end of the stylet 991. The stylet 991 may be inserted into the cannula 901 until the stylet cap 992 abuts the cannula hub 981. The cannula hub 981 may have a portion that complements the stylet cap 992 such that upon contact, the two components have a releasably secure pressure fit.

Next, anesthesia is injected through the cannula 901 and out of the one or more fenestrations 941. The anesthesia is administered to the target nerve 131 for the purpose of blocking nociception and pain during the ablation. Once the anesthesia has been administered to the anesthesia target area 961, a wire probe (not shown) is inserted through the cannula 901. Once in contact with the active tip 921, the wire probe is heated to a specified temperature in order to create a circumferential lesion around the active tip 921, depicted in FIG. 9 as the ablation target area 971.

In accordance with aspects of the disclosure, the cannula 901 of the present disclosure may be used to provide anesthesia to an anesthesia target area 961 around a circumference of the active tip 921. As a result, there is substantial overlap between the anesthesia target area 961 and an ablation target area 971. Accordingly, the entirety of the ablation target area 971 has received anesthesia.

In FIG. 9, the anesthesia target area 961 and the ablation target area 971 are both depicted as having the same general shape. Moreover, the anesthesia target area 961 is slightly larger than the ablation target area 971. However, it will be understood that the shape and size of the anesthesia target area 961 and the ablation target area 971, respectively, may vary depending on the characteristics of the anesthesia, the size and shape of the active tip 921, or the amount of electrical current provided thereto.

The terminology used herein is for the purpose of describing particular embodiments only and not to limit any embodiments disclosed herein. As used herein, the singular forms “a”, “an” and “the” are intended to include the plural forms as well, unless the context clearly indicates otherwise. It will be further understood that the terms “comprises”, “comprising”, “includes” and/or “including”, when used herein, specify the presence of stated features, integers, steps, operations, elements, and/or components, but do not preclude the presence or addition of one or more other features, integers, steps, operations, elements, components, and/or groups thereof. Similarly, the phrase “based on” as used herein does not necessarily preclude influence of other factors and should be interpreted in all cases as “based at least in part on” rather than, for example, “based solely on”.

It will be understood that terms such as “top” and “bottom”, “forward” and “backward”, “left” and “right”, “vertical” and “horizontal”, “distal” and “proximal”, etc., are relative terms used strictly in relation to one another, and do not express or imply any relation with respect to gravity, a patient being treated, a manufacturing device used to manufacture the components described herein, or to any other device used in a medical procedure.

It should be understood that any reference to an element herein using a designation such as “first,” “second,” and so forth does not generally limit the quantity or order of those elements. Rather, these designations may be used herein as a convenient method of distinguishing between two or more elements or instances of an element. Thus, a reference to first and second elements does not imply that there are only two elements and further does not imply that the first element must precede the second element in some manner. Also, unless stated otherwise a set of elements may comprise one or more elements. In addition, terminology of the form “at least one of A, B, or C” or “one or more of A, B, or C” or “at least one of the group consisting of A, B, and C” used in the description or the claims means “A or B or C or any combination of these elements.”

While the foregoing disclosure shows various illustrative aspects, it should be noted that various changes and modifications may be made to the illustrated examples without departing from the scope defined by the appended claims. The present disclosure is not intended to be limited to the specifically illustrated examples alone. For example, unless otherwise noted, the functions, steps, and/or actions of the method claims in accordance with the aspects of the disclosure described herein need not be performed in any particular order. Furthermore, although certain aspects may be described or claimed in the singular, the plural is contemplated unless limitation to the singular is explicitly stated.

What is claimed is:

1. A radiofrequency cannula, comprising:
a cannula body configured to deliver anesthetic and/or electric current therethrough; and
an active tip located distally with respect to the cannula body, wherein the active tip comprises one or more fenestrations configured to pass anesthetic laterally from at least one side of the active tip.

2. The radiofrequency cannula of claim 1, wherein the active tip further comprises a closed distal end configured to prevent passage of anesthetic distally to the active tip.

3. The radiofrequency cannula of claim 1, wherein the active tip further comprises a distal opening configured to pass anesthetic distally to the active tip.

4. The radiofrequency cannula of claim 1, wherein a distal end of the active tip is sharp.

5. The radiofrequency cannula of claim 4, wherein the distal end of the active tip is cone-shaped.

6. The radiofrequency cannula of claim 4, wherein the distal end of the active tip is chisel-shaped.

7. The radiofrequency cannula of claim 1, wherein a distal end of the active tip is blunt.

8. The radiofrequency cannula of claim 1, wherein the one or more fenestrations have one or more shapes selected from a group consisting of:
ellipses;
triangles;
slots;
rectangles.

9. The radiofrequency cannula of claim 8, wherein the one or more fenestrations are linearly arranged on one side of the active tip.

10. The radiofrequency cannula of claim 8, wherein the one or more fenestrations are arranged around a circumference of the active tip.

11. A method, comprising:
maneuvering a cannula body having an active tip located distally with respect to the cannula body to a treatment site having a target area, wherein the active tip comprises one or more fenestrations configured to pass anesthetic laterally from at least one side of the active tip;
delivering anesthetic laterally to the target area via the cannula body and through the one or more fenestrations;
inserting a radiofrequency probe through the cannula body such that it contacts the active tip;
activating the radiofrequency probe to ablate the target area

12. The method of claim 11, wherein the active tip further comprises a closed distal end configured to prevent passage of anesthetic distally to the active tip.

13. The method of claim 11, wherein the active tip further comprises a distal opening configured to pass anesthetic distally to the active tip.

14. The method of claim 11, wherein a distal end of the active tip is sharp.

15. The method of claim 14, wherein the distal end of the active tip is cone-shaped.

16. The method of claim 14, wherein the distal end of the active tip is chisel-shaped.

17. The method of claim 11, wherein a distal end of the active tip is blunt.

18. The method of claim 11, wherein the one or more fenestrations have one or more shapes selected from a group consisting of:
ellipses;
triangles;
slots;
rectangles.

19. The method of claim 18, wherein:
the one or more fenestrations are linearly arranged on one side of the active tip;
the maneuvering comprises turning the cannula body such that the linear arrangement of the one or more fenestrations is in contact with and/or proximate to the target area; and
the anesthetic is delivered to the target area via the one side of the active tip.

20. The method of claim 18, wherein:
the one or more fenestrations are arranged around a circumference of the active tip;
the maneuvering comprises guiding the cannula body such that a side of the active tip is in contact with and/or proximate to the target area; and
the anesthetic is delivered to the target area via the active tip.

21. A radiofrequency cannula, comprising:
means for passing anesthetic and/or electric current therethrough; and
means for delivering anesthetic and/or electric current located distally with respect to the means for passing, wherein the means for delivering comprises one or more fenestrations configured to pass anesthetic laterally from at least one side of the active tip.

22. The radiofrequency cannula of claim 21, wherein the means for delivering further comprises a closed distal end configured to prevent passage of anesthetic distally to the means for delivering.

23. The radiofrequency cannula of claim 21, wherein the means for delivering further comprises a distal opening configured to pass anesthetic distally to the means for delivering.

24. The radiofrequency cannula of claim 21, wherein a distal end of the means for delivering is sharp.

25. The radiofrequency cannula of claim 24, wherein the distal end of the means for delivering is cone-shaped.

26. The radiofrequency cannula of claim 24, wherein the distal end of the means for delivering is chisel-shaped.

27. The radiofrequency cannula of claim 21, wherein a distal end of the means for delivering is blunt.

28. The radiofrequency cannula of claim 21, wherein the one or more fenestrations have one or more shapes selected from a group consisting of:
ellipses;
triangles;
slots;
rectangles.

29. The radiofrequency cannula of claim 28, wherein the one or more fenestrations are linearly arranged on one side of the means for delivering.

30. The radiofrequency cannula of claim 28, wherein the one or more fenestrations are arranged around a circumference of the means for delivering.
